(19) United States
(12) Patent Application Publication
(10) Pub. No.: US 2008/0200827 A1
(43) Pub. Date: Aug. 21, 2008

(54) Apparatus For Converting Electromyographic (Emg) Signals For Transference to a Personal Computer

(76) Inventors: Charles Dean Cyphery, Albuquerque, NM (US); Lance H. Butler, Albuquerque, NM (US)

Correspondence Address: ROBERT S. LIPTON, ESQUIRE, 201 NORTH JACKSON STREET, P. O. BOX 934, MEDIA, PA, 19063-0934, US

(21) Appl. No.: 11/914,385

(22) PCT Filed: May 11, 2006
(86) PCT No.: PCT/US06/18331

§ 371 Date: Nov. 13, 2007

Related U.S. Application Data
(60) Provisional application No. 60/680,381, filed on May 11, 2005.

(51) Int. Cl. A61B 5/0488

(52) U.S. Cl. 600/546

(57) ABSTRACT

The present invention is a circuit for acquiring EMG signals and transferring it directly to a standard personal computer (PC) by converting the EMG signals to the Universal Serial Bus (USB) interface. Once the EMG signals have been stored on the PC and made available to a doctor or other clinician, they may be displayed, tested, and/or manipulated to determine the relative health of the muscle that generated the original EMG signals.

### CROSS-REFERENCE TO RELATED APPLICATION

The present application claims the benefit under all applicable U.S. statutes, including 35 U.S.C. §119(e), to U.S. Provisional Application No. 60/680,381 Filed May 11, 2005, titled Apparatus for Converting Electromyographic Signals for Transferrance to a Personal Computer, in the names of Charles Dean Cyphery and Lance H. Butler, which provisional application claims benefit under all applicable U.S. statutes to U.S. application Ser. No. 10/504,301 filed Aug. 9, 2004, titled Comprehensive Neuromuscular Profiler, which application claims the benefit of priority to PCT Application No. PCT/US2004/22210.

This application incorporates by reference to U.S. Provisional Application. No. 60/680,381, U.S. application Ser. No. 10/504,301, and PCT/US2004/22210, as if all three applications were fully set forth herein.

### COMPUTER PROGRAM LISTING

Submitted herewith is a compact disc (1) which includes a computer listing of a program that may be used in connection with the apparatus disclosed herein. The compact disc contains one file, title “CNMP Source.11b”, which is the source code for the Comprehensive Neuromuscular Profiler invention (disclosed in U.S. application Ser. No. 10/504,301) and includes the LabVIEW program that is used to initialize the USB portion of the circuit board disclosed herein.

This application incorporates by reference the entire program and files on said compact disc.

### FIELD OF THE INVENTION

This invention relates generally to a method and apparatus for monitoring the condition of muscles in a muscle group by the sensing and analysis of electromyographic (EMG) signals derived from electrodes positioned close to the muscle group and, more particularly, to an improved apparatus for converting EMG signals into data to be used with a personal computer so that the EMG data can be stored, reviewed, monitored and assessed.

### BACKGROUND OF THE INVENTION

The discovery of the presence of electromyographic (EMG) signals in the muscles of humans, and the change of these signals with muscle activity, spawned development of dedicated electronic devices and techniques for monitoring those signals for the evaluation of the muscles. The EMG signals given off by the muscles are relatively weak (on the order of microvolts) and it is important that the devices used to monitor and record the EMG signals do not introduce noise thereby making it impossible to interpret the signals.

Human musculature involves many hundreds of muscles in various muscle groups, which interact to provide skeletal support and movement. Recent developments in the field of EMG analysis have concentrated on the techniques and/or devices for monitoring the signals of a specific muscle or group of muscles. For example, U.S. Pat. No. 6,532,383 to Maloney et al. discloses an apparatus for detecting and interpreting EMG signals produced by the tongue; U.S. Pat. No. 6,411,843 to Zarychta discloses an apparatus particularly designed to detect EMG signals produced by the diaphragm; and U.S. Pat. No. 6,004,312 to Finneran et al. discloses an apparatus particularly designed to detect EMG signals produced by a muscle group (e.g., back muscles).

The size of the patient's muscle, range and dynamics of motion of the patient's muscle, the strength of the patient's muscles, and the electrical characteristics of the muscles provide information useful to a clinician making treatment decisions for a patient. The same information also may be useful to determine the existence, severity or cause of an injury and whether an injury is acute or chronic for purposes of determining questions of insurance or other liability.

Soft tissue injuries and pathology may occur in any area of the body and may include repetitive stress injuries, injuries to muscles, myofascial injuries, damage to vertebral disks, radiculopathy, and others. These injuries may be difficult to diagnose and hence may be difficult to treat properly.

The personal computer (PC) is currently the most popular form of computing device. The relatively inexpensive price, exceptional power, and availability of programs has led to the purchase and placement of a PC on virtually every desk in private industries, government agencies, research facilities, and universities. In fact, nearly every home in the U.S. has a PC or access to a PC through a school or a local library. With recent technological advancements, especially with the microprocessor, a PC can handle many of the functions that used to be reserved for work stations, or even main-frames.

Many standards have been developed expressly for the PC. For example, the SoundBlaster sound card standards, the PCI express bus, PCI bus, the AGP bus, and the universal serial bus (USB) interface, Firewire interface, etc. all were originally developed for use with the PC.

As with many technologies, the ability to input, record, display, analyze, and manipulate data with a PC is a useful feature. However, because of the complexity of the muscle structure, the number of electrodes/signals needed to acquire useful signals, and the general difficulty in obtaining reliable EMG signals in the first place (preferably in a non-invasive mode), obtaining a useful definition of the muscle activity in a reasonable amount of time and in an economical manner is still subject to current development.

Presently, there are no known devices that allow a practitioner to easily input and record EMG signals to a PC. At the very least, a dedicated apparatus is needed to convert and store the relatively weak EMG signals into a format usable by a clinician. The cost and complexity of such an apparatus are both relatively high. Accordingly, there is a need to quickly obtain EMG signals and store them on a PC for further analyzation and consideration.

### SUMMARY OF THE INVENTION

The present invention is an apparatus consisting of a circuit board that allows a user to quickly and reliably acquire EMG signals and convert them into a format that allows the data to be stored on a PC for display and later manipulation. With higher speeds of operation and greater computing capacity, the capability for handling and operating upon a multiplicity of signals in a reasonable evaluation period has become feasible.

The present invention provides an apparatus for acquiring EMG signals from a patient and converting the EMG signals into a format for reading and storage on a personal computer. This apparatus includes an input for acquiring a plurality of EMG signals from sensors attached to a patient, a means for conditioning the acquired EMG signals, a means for converting the conditioned EMG signals to digital signals, a means for isolating the digital signals from the acquired signals, a means for temporarily storing the digital signals and a means for outputting the stored signals in a serial format for inputting into a personal computer.

The input for receiving the EMG signals includes a channel acquisition board which can handle inputs from a plurality of EMG sensor leads and a plurality of strain gauge inputs. Each EMG sensor lead consists of a single channel and has two sensors attached in order to measure the differential voltage. In the preferred embodiment the channel acquisition board consists of 18 channels for EMG sensor leads and six strain gauge input channels. These inputs are connected to the channel acquisition board preferably by a ribbon cable through two 40-pin connectors.

The means for conditioning the acquired EMG signals is carried out through the use of filtering and amplifying circuitry so that the signals can be recognized by the analog-to-digital converters. This conditioning is very important in order to ensure signal integrity, as the signals read from the patient are on the order of a microvolt and must be filtered and amplified before being recognized by any readily-available circuitry that can be used to store the data digitally on a computer. In addition, because the acquired EMG signals are so small, noise is a major factor in acquiring accurate EMG data from the patient's muscle.

The means for converting the conditioned EMG signals to digital signals is carried out through the use of analog-to-digital converters. There are three analog-to-digital converters which receive the conditioned EMG signals and convert the analog signals to digital signals.

The means for isolating the digital signals from the acquired signals is carried out through the use of optical isolators. These optical isolators ensure that damaging signals cannot reach either side of the circuit board which could cause damage to individual components.

The means for temporarily storing the digital signals is carried out through the use of memory management buffers. These memory management buffers act to meter the flow of information through the circuit.

The means for outputting the stored signals in a serial format for inputting into a personal computer is carried out using complex programmable logic devices (or a microprocessor) and a USB connector. The complex programmable logic devices are active components that are programmed (e.g., in the JTAG programming language) with algorithms for managing and manipulating the data and to put it into formats required for the circuitry of the apparatus. The USB connector is the mating connector that may be plugged directly into a USB port found on virtually all standard PCs.

The present invention also provides a method for acquiring EMG signals from a patient and converting the EMG signals into a format for reading and storage on a personal computer. This method includes acquiring a plurality of EMG signals from sensors attached to a patient, conditioning the acquired EMG signals, converting the conditioned EMG signals to digital signals, isolating the digital signals from the acquired signals, storing the digital signals and a means for outputting the stored signals in a serial format for inputting into a personal computer.

### BRIEF DESCRIPTION OF THE DRAWINGS

The accompanying drawings, which are incorporated in and form a part of the specification, illustrate the embodiments of the present invention and, together with the following description, serve to explain the principles of the invention. For the purpose of illustrating the invention, there are shown in the drawings embodiments which are presently preferred, it being understood, however, that the invention is not limited to the specific instrumentality or the precise arrangement of elements or process steps disclosed.

In the drawings:

FIG. 1 is a schematic block diagram of a circuit designed to acquire EMG signals from sensors attached to a patient and convert said signals into a digital signal in accordance with the present invention;

FIG. 2 is a schematic block diagram of a circuit designed to take the digital signal generated by the circuit illustrated in FIG. 1 and convert the digital signal into a USB format for direct connection to a PC;

FIGS. 3A-3H are a graphical representation of the LabVIEW program used to initiate the USB portion of the circuit board disclosed in FIGS. 1 and 2.

### DETAILED DESCRIPTION OF THE PREFERRED EMBODIMENT

In describing a preferred embodiment of the invention, specific terminology will be selected for the sake of clarity. However, the invention is not intended to be limited to the specific terms so selected, and it is to be understood that each specific term includes all technical equivalents that operate in a similar manner to accomplish a similar purpose.

Preferred embodiments of the present invention will now be described in detail with reference to the accompanying drawings in which an apparatus for acquiring electromyographic (EMG) signals derived from electrodes positioned close to the muscle group in accordance with the present invention is generally indicated at 10.

EMG signals and their relation to muscle functions are well understood at the current state of investigations. Muscles are controlled by nerves, the latter transmitting an electrical signal to a particular muscle and causing contraction thereof. The muscle itself is a volume conductor reacting to the signal of the associated nerve. There is a voltage change that occurs when a muscle contracts creating an electric potential that is directly proportional to the strength of contraction and that can be captured from the external surface area of the patient.

As is known in the art, a transducer (e.g., a sensor pad, electrode, etc.) is a device for collecting electromyographic (EMG) signals from the the desired muscle on the patient's body. The transducer is usually placed on the patient's skin proximate to the muscle which is to be analyzed.

A typical sensor pad (not shown) is a flat rectangular piece of siliconized rubber, approximately 0.062 inches thick. One source for this type of sensor pad is Fairprene Industrial Products, Inc. of Fairfield, Conn.

Although the apparatus and method described herein deals mainly with acquiring EMG signals from primary muscles without invasive procedures, it would be understood by one skilled in the art after reading the present disclosure that this invention can be adapted for use with any muscle in which a transducer can be used to obtain an EMG signal (e.g., the diaphragm).

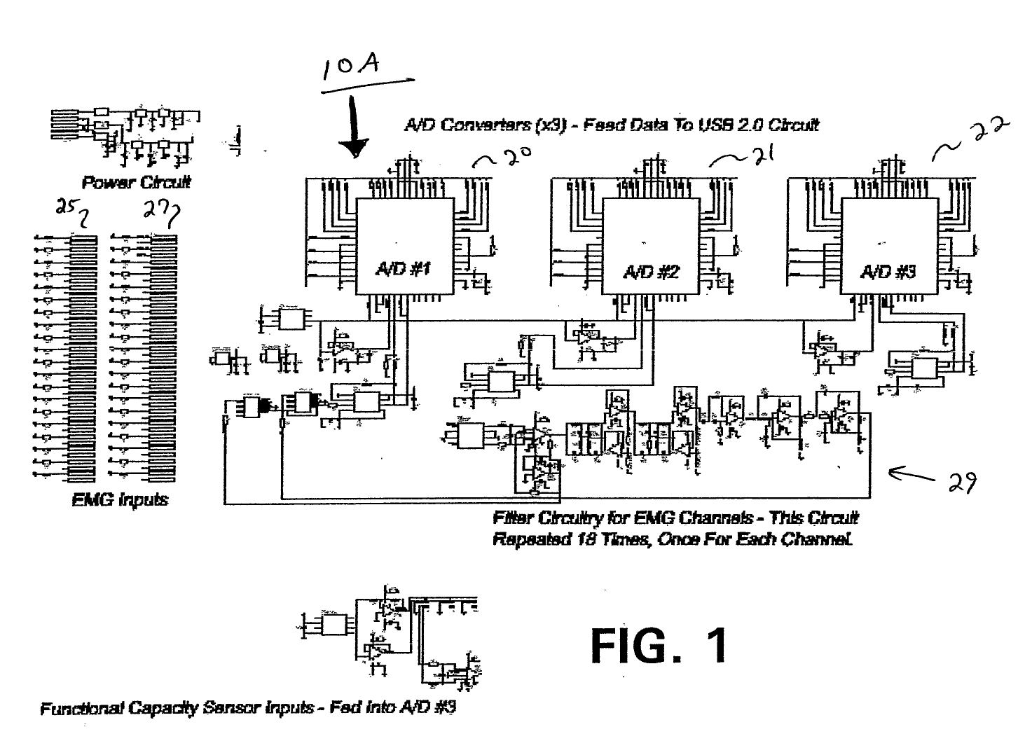

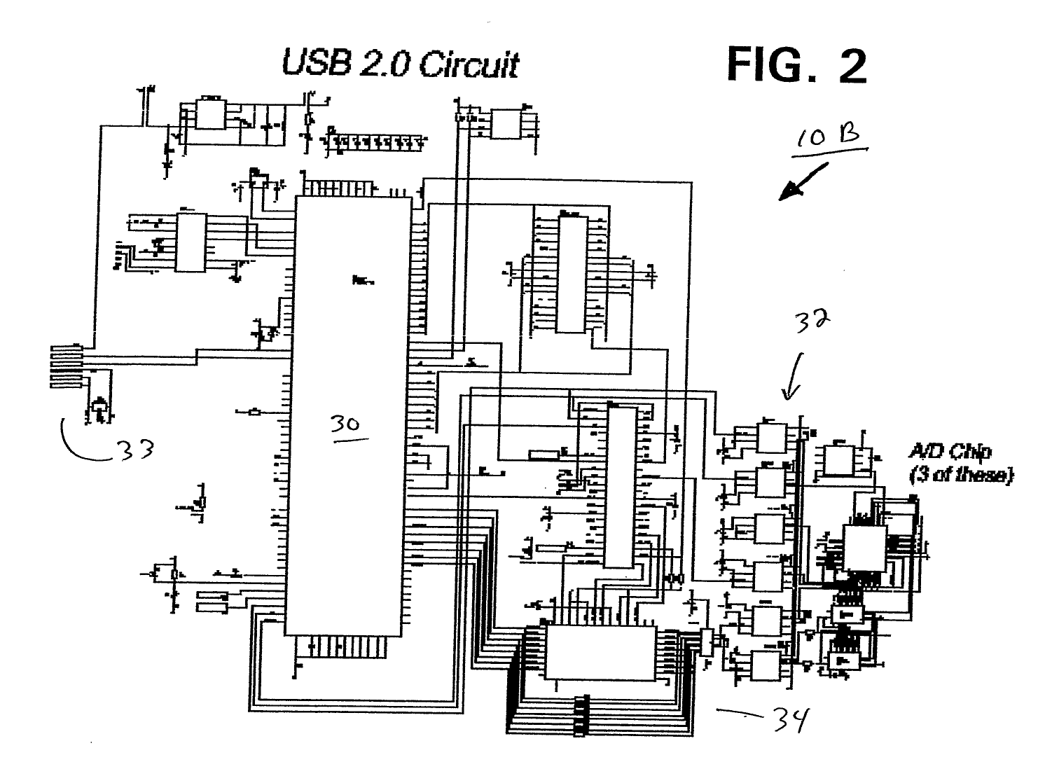

Referring now to the drawings, and initially to FIGS. 1 and 2, there is shown in schematic form a circuit 10 for acquiring EMG data and transferring it directly to a standard personal computer (PC) utilizing the Universal Serial Bus (USB) interface. It is understood that such a circuit is commonly placed on a circuit board and reference to the apparatus of a circuit board is occasionally made herein.

FIG. 1 discloses the schematic diagram for the EMG acquisition portion 10A of the circuit board. The principal task of this portion 10A of the board is to accept input from eighteen pairs of EMG sensors placed on the human body. EMG signals are differential signals and, therefore, each channel requires two inputs (i.e., a pair of inputs for each EMG sensor). Each pair of leads is designated as a single channel and as such, this is an eighteen channel acquisition board. These eighteen pairs of leads are input to the board preferably by means of a ribbon cable through two 40-pin connectors 25, 27. Of course, more or less pairs of sensors may be used depending on the size and location of the muscle to be analyzed, the type of sensor needed, etc. may determine the final number of sensors used.

There are two connectors for the EMG signals; each connector handling nine channels of data. There is a single ground lead used for each grouping of nine channels. The ground lead is grounded to the patient's body, preferably to a bone closest to the muscle/muscle group under observation.

The EMG signals from the muscles are on the order of microvolts. The data acquisition portion 10A of the subject apparatus conditions the EMG signals via filtering and amplifying circuitry 29, so that the signals can be recognized by analog-to-digital converters (ADC) 20, 21, 22 as illustrated in FIG. 1. The conditioning is very important in order to ensure signal integrity, as the signals read from the muscle of the human patient are on the magnitude of a microvolt and must be filtered and amplified before being converted into a digital signal. In addition, since the original EMG signals are so small in amplitude, noise is a major factor in acquiring accurate EMG data from the muscle.

The circuit board contains eighteen identical circuits each of which handle one channel of data, or one pair of EMG leads. After conditioning each of the channels of EMG data, the signal amplitudes are between −2.5 volts (V) and +2.5V and are an analog representation of the EMG signals generated by the patient's muscle. In addition to the eighteen EMG conditioning circuits, the board also has provisions to accept and condition the inputs from six strain gauge outputs.

Currently three of these strain gauge inputs are not used and are reserved for future expansion; the other three are used for making functional capacity measurements such as measuring grip strength, pinch strength and a load cell used for pulling on and all three return signals representative of the force applied. All three of these inputs require a +2.5V and a −2.5V input which is used as an excitation voltage to activate the circuit in the gauge and then when a force is a applied to the gauge, the signal returned ranges between 0V and 2.5V where 2.5V represents the full scale range of the gauge in pounds. Each strain gauge preferably has its own cable and connector for connecting to the circuit board.

All twenty-four of these inputs are then fed into three separate ADC circuits 20 where the data is converted from their initial analog format to digital format. The data, acquisition portion 10A then passes the EMG signals through to the data conversion portion 10B of the circuit board.

Referring again to FIG. 2, the digital data is then sent in a serial stream to optical isolators 32 on the USB conversion portion 10B of the circuit board. The optical isolators 32 insure that damaging signals cannot reach either side of the board which could cause damage to individual components. The USB circuitry then acts to further format this data into a format suitable for transferring to a standard PC.

After the signals pass through the opto-isolators 32, they are passed to memory management buffers 34, three Complex Programmable Logic Devices (CPLD) 30 and finally to a USB connector 33. The USB connector is the mating connector that may be plugged directly into a USB port found on virtually all standard PCs or connected via a USB cable.

Although the present invention utilizes CPLDs 30, a person skilled in the art could replace the CPLDs with Field Programmable Gate Arrays, microprocessors, or other Programmable Devices. The CPLDs can be programmed to perform different tasks at different times. However, the CPLDs are not as complex nor as expensive as a microprocessor.

The CPLDs 30 control the output of the EMG data stored in the management buffers 34 in accordance with programs stored on chips on the circuit board and in accordance with USB 2.0 format. Of course, the chips can be programmed to control the output of EMG data in accordance with any format readable by a personal computer.

Once the EMG data has been transferred to the computer, a physician can monitor the data, manipulate the data, store the data, compare the data for future analysis and/or comparisons. The personal computer can perform a number of tests on the raw data. Also, the personal computer can store large amounts of data very cheaply.

The CPLDs 30 are active and intelligent components in that they are programmed with algorithms for managing and/or manipulating the data to put it into formats required for circuitry farther down the line. These components are programmed using the Joint Test Action Group (JTAG) programming language and must be properly programmed in order for the circuit board to function properly and to convert the EMG signals into the USB format for communication with a personal computer. In this embodiment, the JTAG program is loaded during the assembly of the circuit board 10. Of course the program used will depend on the type of active components selected and the chip's underlying process technology. If the CPLDs or FPGAs require ABEL or PALASM or any of a number of similar programming languages, they will be used instead of JTAG.

The program handles many functions including the flow of data being handled by the analog-to-digital converters 20, 21, 22 shown in FIG. 1. In this embodiment, the JTAG program directs the processor to send commands across the opto-isolators 32 to the AD converters to meter the data coming from the data acquisition circuit 10A.

In one preferred embodiment, the JTAG program can be burned onto a chip once during the assembly of the board, or the program can be stored on an EPROM and loaded every time the board is initialized (e.g., the system is turned ON, or RESET). The choice is a design parameter and depends on various factors.

The USB circuit including the CPLDs must undergo an initialization sequence. The initialization sequence can be handled through either software or hardware. If handled using software, any of a number of languages may be used which software is stored on the circuit board. In a preferred embodiment, the LabVIEW language is used to program the initialization sequence. Any language the board designer wishes to use to communicate with the circuit board, (e.g., C, C++, Visual Basic, Visual C++, MatLab, etc.) can be used for the initialization sequence.

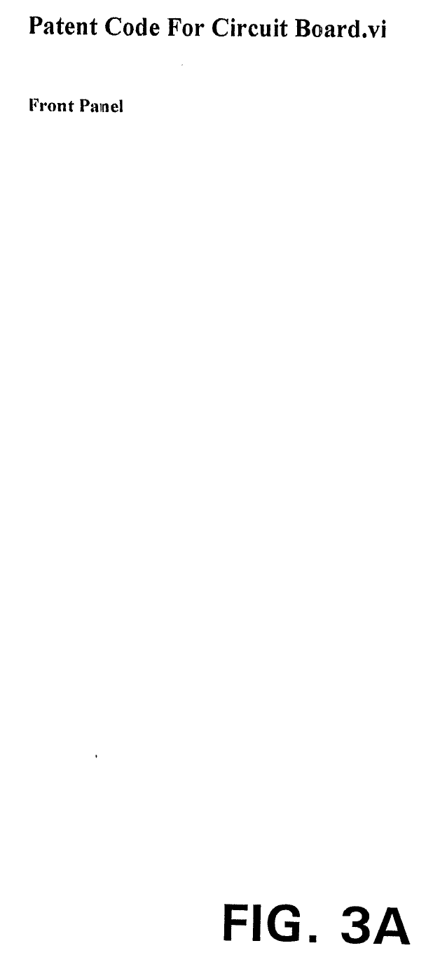

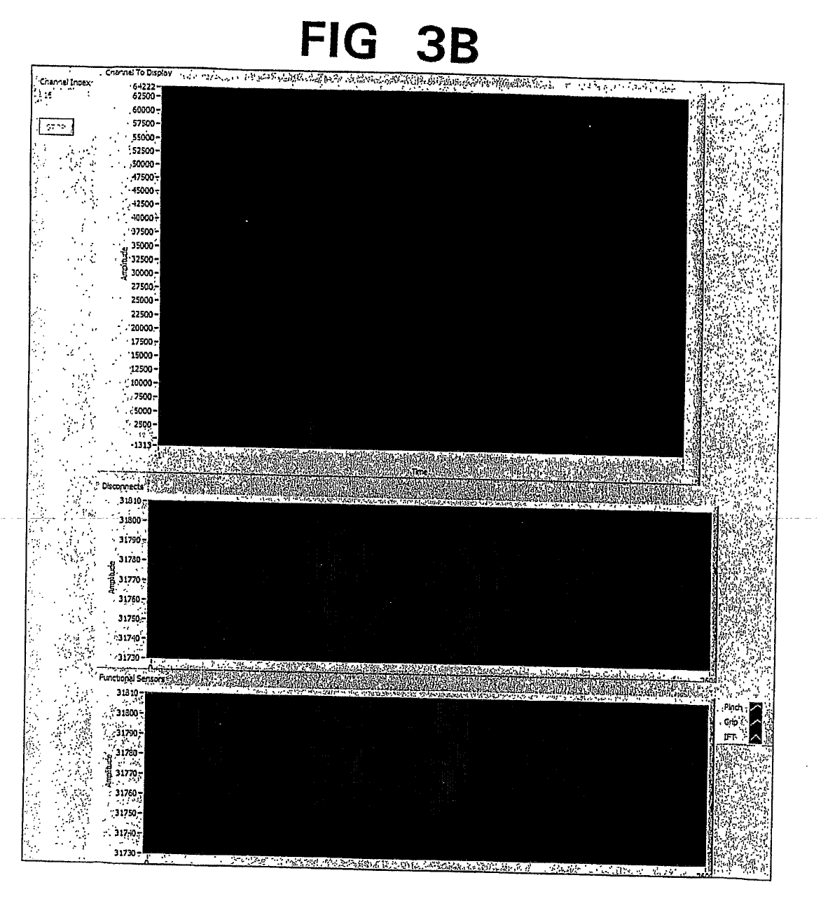

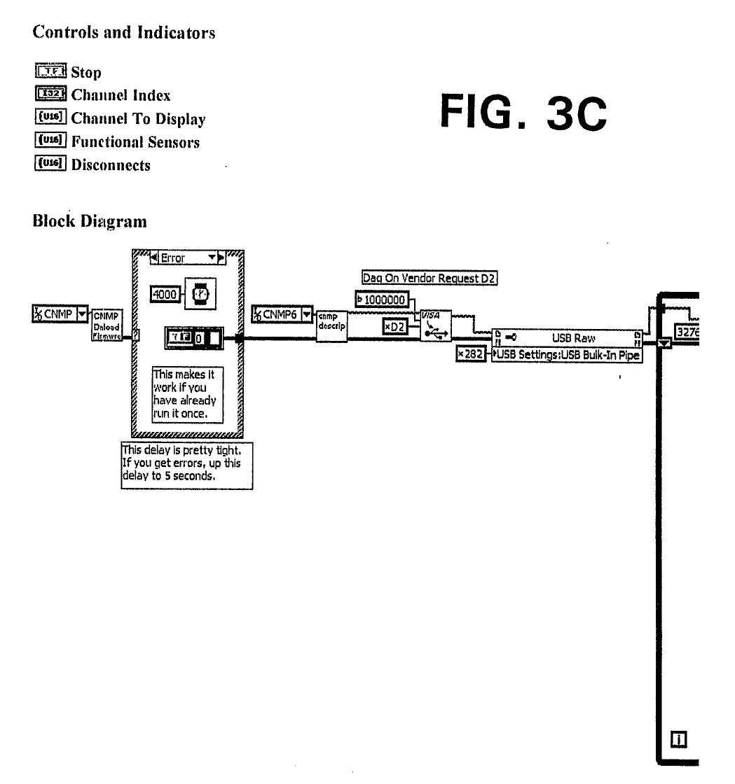

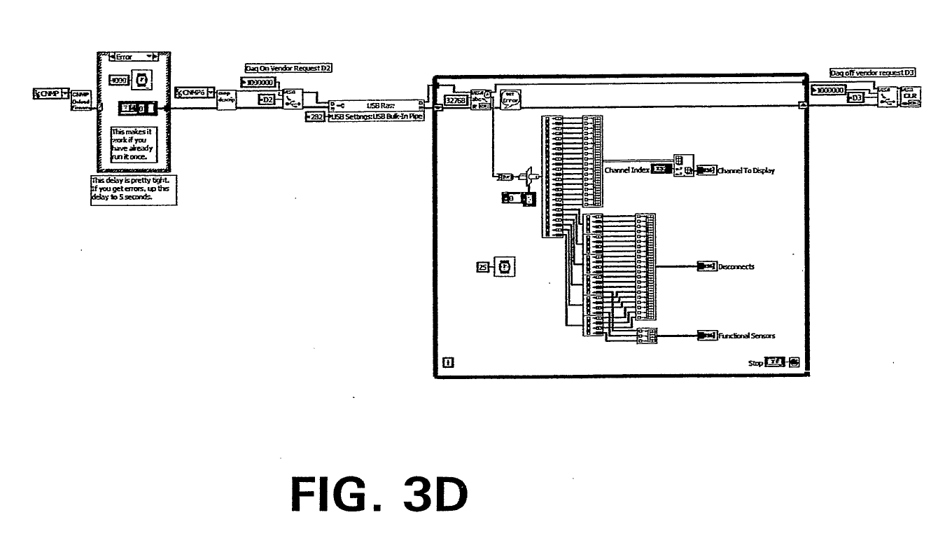

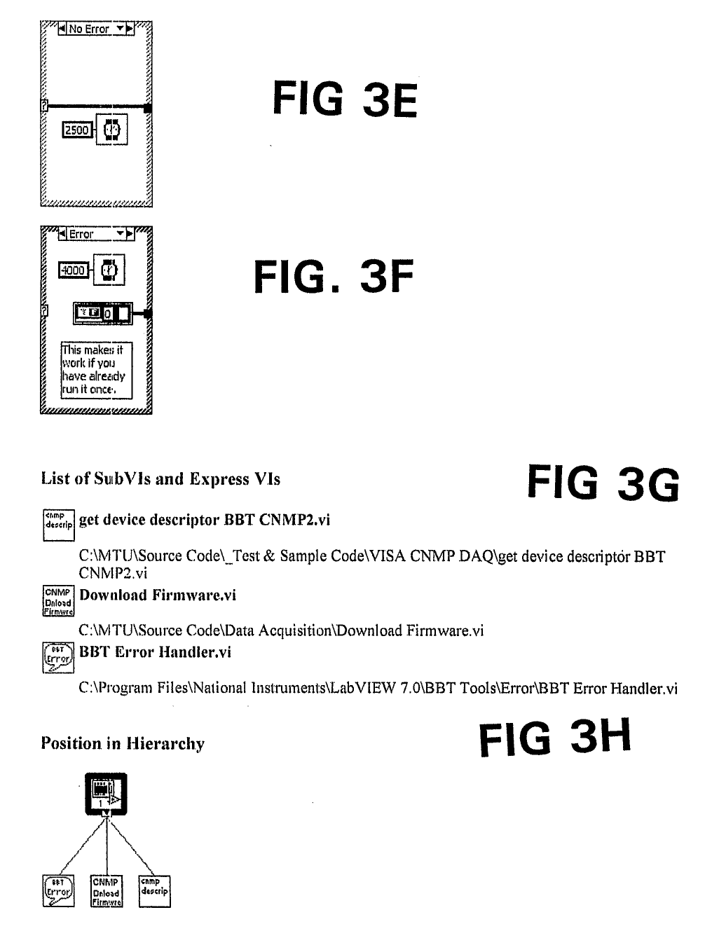

Referring now to FIGS. 3A-3H, the LabVIEW program used to operate the circuit board is presented in graphical form. A copy of the LabVIEW program is contained on the enclosed compact disc.

The PC must be loaded with and running driver software that has the ability to communicate with the EMG sensor board 10. In the preferred embodiment, the circuit board can be designed as a plug-n-play installation for a PC running, for example, the WINDOWS®), MAC® or LINUX operating systems. The board manufacturer can develop the driver software just like any piece of hardware developed for a PC (e.g., a printer, a video card, etc.).

In a preferred embodiment, the circuit board 10 can be manufactured as a multi-layer board built to very tight tolerances to ensure proper signal integrity in the USB circuitry and also to distribute various signals to common points on the board. The majority of the components placed on the board are passive components in that they act on signals that are fed into them in a predefined and fixed fashion. Other components, in particular the processor, are active components.

It would be apparent to one skilled in the art after reading this disclosure that other circuitry and programming language can be used to carry out the conversion of the digital EMG signals into a USB signal for direct connection to a PC than those shown in FIG. 2. Also, once the present invention acquires the EMG signal and converts it into a digital signal, the CPLDs 30 and the active components can be modified to allow communication with a computer via the standard serial connector, Firewire, or other means that has been or may be employed to communicate with a computer.

The present invention may be adapted for use with various devices that acquire EMG signals and process same. In one example, the present invention may be used with the Comprehensive Neuromuscular Profiler invention disclosed in U.S. patent application Ser. No. 10/504,301 filed Aug. 9, 2004. The source code of the software developed to operate the Comprehensive Neuromuscular Profiler (including the program that is used by the circuit board that converts EMG signals into a USB signal) is included on the attached compact disc.

Although this invention has been described and illustrated by reference to specific embodiments, it will be apparent to those skilled in the art that various changes and modifications may be made which clearly fall within the scope of this invention. The present invention is intended to be protected broadly within the spirit and scope of the appended claims.

We Claim:

1. An apparatus for converting electromyographic (EMG) signals from a patient into a format useable by a computer, the apparatus comprising:
a.) an input for receiving one or more EMG signals from a sensor attached to the patient;
b.) means for conditioning said EMG signals;
c.) means for converting said conditioned EMG signals to digital signals;
d.) means for isolating said digital signals from said conditioning means and from said converting means;
e.) means for temporarily storing said digital signals; and
f) means for outputting said stored digital signals in a serial format for inputting to the personal computer.

2. The apparatus of claim 1 wherein the serial format is universal serial bus (USB) 2.0.

3. The apparatus of claim 1 wherein the serial format is a standard identified as IEEE-1394 (usually referred to as Firewire).

4. The apparatus of claim 1 wherein the means for outputting comprises a Complex Programmable Logic Device (CPLD), and an EPROM for storing instructions electrically, said EPROM communicating with said CPLD.

5. The apparatus of claim 1 wherein the means for outputting comprises a microprocessor.

6. The apparatus of claim 1 wherein the means for outputting includes a microprocessor.

7. The apparatus of claim 1 wherein the means for outputting includes a Field Programmable Gate Array (FPGA).

8. The apparatus of claim 1 wherein the conditioning means includes at least one operational amplifiers.

9. The apparatus of claim 1 wherein the means for converting includes a plurality of A/D converters.

10. The apparatus of claim 1 wherein the means for isolating includes a plurality of optical isolators.

11. The method of converting EMG signals from a patient to a format readable by a personal computer, the method comprising the steps of:
a.) acquiring the EMG signals from the patient;
b.) conditioning said acquired EMG signals;
c.) converting said conditioned EMG signals into digital signals;
d.) isolating said digital signals from said conditioned and acquired signals;
e.) storing the isolated digital signals; and
f.) outputting said isolated signals in a controlled format readable by a computer.

12. An apparatus for converting electromyographic (EMG) signals from a patient into a computer-readable format, the EMG signal being sensed by at least one transducer, the apparatus comprising:
a.) a plurality of operational amplifier circuits for receiving the EMG signals from the patient, said operational amplifiers also conditioning and amplifying said EMG signals;
b.) a plurality of analog-to-digital circuits for converting said conditioned EMG signals to digital signals, said digital signals representing the EMG data sensed by the transducers;
c.) a plurality of optical isolators for isolating the digital signals from the operational amplifier circuit and the analog-to digital circuit;
d.) a buffer for temporarily storing said digital signals;
e.) a Complex Programmable Logic Device (CPLD) for controlling the buffers and outputting the data in a specific format readable by a computer; and
f.) active component that stores instructions for the CPLD to process and to manipulate said digital signals into a format readable by a computer.

13. The apparatus of claim 12 wherein the operational amplifiers circuits condition and amplify the EMG signal by scaling the signal's amplitude to between −2.5 volts and 2.5 volts.

14. The apparatus of claim 12 wherein said instructions are in the Joint Test Action Group (JTAG) programming language.

15. The apparatus of claim 12 wherein said format readable by a computer is USB 2.0.

16. The apparatus of claim 12 further comprising a USB connector for electrically connecting the apparatus to the computer.

17. The apparatus of claim 12 further comprising an EPROM that is electrically connected to the CPLD and provides initinstructions to the Complex Programmable Logic Devices (CPLD) upon start-up or reboot.
